(19) United States
(12) Patent Application Publication
(10) Pub. No.: US 2016/0280631 A1
(43) Pub. Date: Sep. 29, 2016

(54) ESTER COMPOUNDS INCLUDING TRIESTERS HAVING TERMINAL VICINAL ACYL GROUPS

(71) Applicant: BIOSYNTHETIC TECHNOLOGIES, LLC., Irvine, CA (US)

(72) Inventor: Jeremy FOREST, Honolulu, HI (US)

(21) Appl. No.: 15/073,540

(22) Filed: Mar. 17, 2016

Related U.S. Application Data
(60) Provisional application No. 62/138,289, filed on Mar. 25, 2015.

(51) Int. Cl. C07C 69/24; C10M 169/04; C10M 129/72

(52) U.S. Cl. CPC C07C 69/24; C10M 129/72; C10M 169/04; C10M 2207/282; C10N 2030/02

(57) ABSTRACT

Provided herein are certain esters, including those of the Formula I:

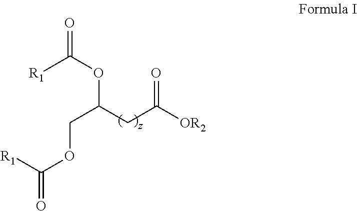

wherein z is an integer selected from 0 to 15; R₁, independently for each occurrence, is an optionally substituted alkyl that is saturated or unsaturated, and branched or unbranched; and R₂ is selected from hydrogen and optionally substituted alkyl that is saturated or unsaturated, and branched or unbranched. Hydroxy compounds are also described herein, which may be suitable end products, or serve as intermediates, to provide the desired ester products. Also described are compositions containing certain esters (e.g., triesters) and methods of making such esters and compositions thereof.

### FIELD

The present disclosure relates to certain ester compounds, such as triesters comprising vicinal acyl groups. The triester compounds described herein may be useful as lubricant base stocks or additives to lubricant formulations.

### BACKGROUND

A variety of commercial uses for fatty esters such as triglycerides have been described. When used as a lubricant, for example, fatty esters can provide a biodegradable alternative to petroleum-based lubricants. However, naturally-occurring fatty esters are typically deficient in one or more areas, including hydrolytic stability and/or oxidative stability.

### SUMMARY

Described herein are ester compounds including triester compounds, triester-containing compositions, and methods of making the same. In certain embodiments, such compounds and/or compositions may be useful as base oils and lubricant additives. In certain embodiments, the compounds comprise at least one compound selected from Formula I:

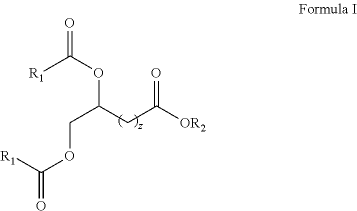

wherein

z is an integer selected from 0 to 15;

R₁, independently for each occurrence, is an optionally substituted alkyl that is saturated or unsaturated, and branched or unbranched; and

R₂ is selected from hydrogen and optionally substituted alkyl that is saturated or unsaturated, and branched or unbranched.

Also described herein are certain compounds which may be useful as lubricants, additives, or intermediates to such compounds. In certain embodiments, the compounds are selected from those represented by Formula II:

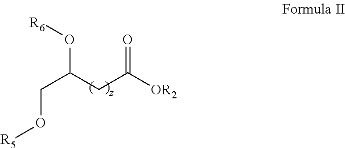

wherein

z is an integer selected from 0 to 15;

R₅ and R₆ are independently selected from hydrogen, —C(O)R₁, and an optionally substituted alkyl that is saturated or unsaturated, and branched or unbranched;

R₁ is, independently for each occurrence, an optionally substituted alkyl that is saturated or unsaturated, and branched or unbranched; and

R₂ is selected from hydrogen and optionally substituted alkyl that is saturated or unsaturated, and branched or unbranched.

### DETAILED DESCRIPTION

The compounds and compositions described herein may exhibit superior properties when compared to other lubricant additives and compositions. Exemplary compositions include, but are not limited to, coolants, fire-resistant and/or non-flammable fluids, dielectric fluids such as transformer fluids, greases, drilling fluids, crankcase oils, hydraulic fluids, passenger car motor oils, 2- and 4-stroke lubricants, metalworking fluids, food-grade lubricants, refrigerating fluids, compressor fluids, and plasticized compositions.

The use of lubricants and lubricating fluid compositions may result in the dispersion of such fluids, compounds, and/or compositions in the environment. Petroleum base oils used in common lubricant compositions, as well as additives, are typically non-biodegradable and can be toxic. The present disclosure provides for the preparation and use of compositions comprising partially or fully bio-degradable base oils, including base oils comprising one or more triesters.

In certain embodiments, the lubricants and/or compositions comprising one or more triesters are partially or fully biodegradable and thereby pose diminished risk to the environment. In certain embodiments, the lubricants and/or compositions meet guidelines set for by the Organization for Economic Cooperation and Development (OECD) for degradation and accumulation testing. The OECD has indicated that several tests may be used to determine the “ready biodegradability” of organic chemicals. Aerobic ready biodegradability by OECD 301D measures the mineralization of the test sample to CO₂ in closed aerobic microcosms that simulate an aerobic aquatic environment, with microorganisms seeded from a waste-water treatment plant. OECD 301D is considered representative of most aerobic environments that are likely to receive waste materials. Aerobic “ultimate biodegradability” can be determined by OECD 302D. Under OECD 302D, microorganisms are pre-acclimated to biodegradation of the test material during a pre-incubation period, then incubated in sealed vessels with relatively high concentrations of microorganisms and enriched mineral salts medium. OECD 302D ultimately determines whether the test materials are completely biodegradable, albeit under less stringent conditions than “ready biodegradability” assays.

As used in the present specification, the following words, phrases and symbols are generally intended to have the meanings as set forth below, except to the extent that the context in which they are used indicates otherwise. The following abbreviations and terms have the indicated meanings throughout:

A dash (“-”) that is not between two letters or symbols is used to indicate a point of attachment for a substituent. For example, —C(O)NH₂ is attached through the carbon atom.

“Alkoxy” by itself or as part of another substituent refers to a radical —OR³¹ where R³¹ is alkyl, cycloalkyl, cycloalkylalkyl, aryl, or arylalkyl, which can be substituted, as defined herein. In some embodiments, alkoxy groups have from 1 to 8 carbon atoms. In some embodiments, alkoxy groups have 1, 2, 3, 4, 5, 6, 7, or 8 carbon atoms. Examples of alkoxy groups include, but are not limited to, methoxy, ethoxy, propoxy, butoxy, cyclohexyloxy, and the like.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

Unless otherwise indicated, the term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group comprises from 1 to 40 carbon atoms, in certain embodiments, from 1 to 22 or 1 to 18 carbon atoms, in certain embodiments, from 1 to 16 or 1 to 8 carbon atoms, and in certain embodiments from 1 to 6 or 1 to 3 carbon atoms. In certain embodiments, an alkyl group comprises from 8 to 22 carbon atoms, in certain embodiments, from 8 to 18 or 8 to 16. In some embodiments, the alkyl group comprises from 3 to 20 or 7 to 17 carbons. In some embodiments, the alkyl group comprises 1, 2, 3, 4, 5, 6, 7, 8, 9, 10, 11, 12, 13, 14, 15, 16, 17, 18, 19, 20, 21, or 22 carbon atoms.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered non-aromatic heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can comprise from 5 to 20 carbon atoms, and in certain embodiments, from 5 to 12 carbon atoms. In certain embodiments, an aryl group can comprise 5, 6, 7, 8, 9, 10, 11, 12, 13, 14, 15, 16, 17, 18, 19, or 20 carbon atoms. Aryl, however, does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“Estolide” as used herein may generally refer to a certain oligomeric/polymeric compounds comprising at least one carboxylic group bound to the hydrocarbon backbone (i.e., alkyl residue) of at least one second carboxylic group. Estolides may be naturally occurring or synthetically derived. Exemplary synthetic estolides include, but are not limited to, oligomeric/polymeric compounds comprising two or more fatty acid residues, which may be formed by the addition of one fatty acid to the hydrocarbon backbone of a second fatty acid residue via an addition reaction across a site of unsaturation, or a condensation reaction with a hydroxyl group. Naturally occurring estolides may include esto-glyceride type compounds (e.g., triacylglycerol estolides), such as those found in certain hydroxy-containing triglycerides of the genus lesquerella, mallotus, or trewia. Per this definition, the triesters described herein comprising terminal vicinal acyl groups may be considered estolides. However, unless specified to the contrary, any reference herein to the term “estolide” shall not encompass the triesters comprising terminal vicinal acyl groups described herein.

“Compounds” refers to compounds encompassed by structural Formula I-III herein and includes any specific compounds within the formula whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

For the purposes of the present disclosure, “chiral compounds” are compounds having at least one center of chirality (i.e. at least one asymmetric atom, in particular at least one asymmetric C atom), having an axis of chirality, a plane of chirality or a screw structure. “Achiral compounds” are compounds which are not chiral.

Compounds of Formula I-III include, but are not limited to, optical isomers of compounds of Formula I-III, racemates thereof, and other mixtures thereof. In such embodiments, the single enantiomers or diastereomers, i.e., optically active forms, can be obtained by asymmetric synthesis or by resolution of the racemates. Resolution of the racemates may be accomplished by, for example, chromatography, using, for example a chiral high-pressure liquid chromatography (HPLC) column. However, unless otherwise stated, it should be assumed that Formula I-VII cover all asymmetric variants of the compounds described herein, including isomers, racemates, enantiomers, diastereomers, and other mixtures thereof. In addition, compounds of Formula I-VII include Z- and E-forms (e.g., cis- and trans-forms) of compounds with double bonds. The compounds of Formula I-III may also exist in several tautomeric forms including the enol form, the keto form, and mixtures thereof. Accordingly, the chemical structures depicted herein encompass all possible tautomeric forms of the illustrated compounds.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₃₋₁₂ cycloalkyl or C₅₋₁₂ cycloalkyl. In certain embodiments, a cycloalkyl group is a C₅, C₆, C₇, C₈, C₉, C₁₀, C₁₁, C₁₂, C₁₃, C₁₄, or C₁₅ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“Halogen” refers to a fluoro, chloro, bromo, or iodo group.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which can be aromatic or non-aromatic in which at least one ring atom is a heteroatom. Heteroaryl encompasses 5- to 12-membered aromatic, such as 5- to 7-membered, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments, a heteroaryl group is a 5-, 6-, 7-, 8-, 9-, 10-, 11-, 12-, 13-, 14-, 15-, 16-, 17-, 18-, 19-, or 20-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“Heterocycloalkylalkyl” by itself or as part of another substituent refers to an acyclic alkyl radical in which one of the hydrogen atoms bonded to a carbon atom, typically a terminal or sp³ carbon atom, is replaced with a heterocycloalkyl group. Where specific alkyl moieties are intended, the nomenclature heterocycloalkylalkanyl, heterocycloalkylalkenyl, or heterocycloalkylalkynyl is used. In certain embodiments, a heterocycloalkylalkyl group is a 6- to 30-membered heterocycloalkylalkyl, e.g., the alkanyl, alkenyl, or alkynyl moiety of the heterocycloalkylalkyl is 1- to 10-membered and the heterocycloalkyl moiety is a 5- to 20-membered heterocycloalkyl, and in certain embodiments, 6- to 20-membered heterocycloalkylalkyl, e.g., the alkanyl, alkenyl, or alkynyl moiety of the heterocycloalkylalkyl is 1- to 8-membered and the heterocycloalkyl moiety is a 5- to 12-membered heterocycloalkyl.

“Mixture” refers to a collection of molecules or chemical substances. Each component in a mixture can be independently varied. A mixture may contain, or consist essentially of, two or more substances intermingled with or without a constant percentage composition, wherein each component may or may not retain its essential original properties, and where molecular phase mixing may or may not occur. In mixtures, the components making up the mixture may or may not remain distinguishable from each other by virtue of their chemical structure.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Substituted” refers to a group in which one or more hydrogen atoms are independently replaced with the same or different substituent(s). Examples of substituents include, but are not limited to, —R⁶⁴, —R⁶⁰, —O⁻, —OH, ═O, —OR⁶⁰, —SR⁶⁰, —S⁻, ═S, —NR⁶⁰R⁶¹, ═NR⁶⁰, —CN, —CF₃, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, —C(NR⁶²)NR⁶⁰R⁶¹, —S(O)₂, NR⁶⁰R⁶¹, —NR⁶³S(O)₂R⁶⁰, —NR⁶³C(O)R⁶⁰, and —S(O)R⁶⁰;

wherein each —R⁶⁴ is independently a halogen; each R⁶⁰ and R⁶¹ are independently alkyl, substituted alkyl, alkoxy, substituted alkoxy, cycloalkyl, substituted cycloalkyl, heterocycloalkyl, substituted heterocycloalkyl, aryl, substituted aryl, heteroaryl, substituted heteroaryl, arylalkyl, substituted arylalkyl, heteroarylalkyl, or substituted heteroarylalkyl, or R⁶⁰ and R⁶¹ together with the nitrogen atom to which they are bonded form a heterocycloalkyl, substituted heterocycloalkyl, heteroaryl, or substituted heteroaryl ring, and R⁶² and R⁶³ are independently alkyl, substituted alkyl, aryl, substituted aryl, arylalkyl, substituted arylalkyl, cycloalkyl, substituted cycloalkyl, heterocycloalkyl, substituted heterocycloalkyl, heteroaryl, substituted heteroaryl, heteroarylalkyl, or substituted heteroarylalkyl, or R⁶² and R⁶³ together with the atom to which they are bonded form one or more heterocycloalkyl, substituted heterocycloalkyl, heteroaryl, or substituted heteroaryl rings;

wherein the “substituted” substituents, as defined above for R⁶⁰, R⁶¹, R⁶², and R⁶³, are substituted with one or more, such as one, two, or three, groups independently selected from alkyl, -alkyl-OH, —O-haloalkyl, -alkyl-NH₂, alkoxy, cycloalkyl, cycloalkylalkyl, heterocycloalkyl, heterocycloalkylalkyl, aryl, heteroaryl, arylalkyl, heteroarylalkyl, —O⁻, —OH, ═O, —O-alkyl, —O-aryl, —O-heteroarylalkyl, —O-cycloalkyl, —O-heterocycloalkyl, —SH, —S⁻, ═S, —S-alkyl, —S-aryl, —S-heteroarylalkyl, —S-cycloalkyl, —S-heterocycloalkyl, —NH₂, ═NH, —CN, —CF₃, —OCN, —SCN, —NO, —NO₂, ═N₂, —N₃, —S(O)₂O⁻, —S(O)₂, —S(O)₂OH, —OS(O₂)O⁻, —SO₂(alkyl), —SO₂(phenyl), —SO₂(haloalkyl), —SO₂NH₂, —SO₂NH(alkyl), —SO₂NH(phenyl), —P(O)(O⁻)₂, —P(O)(O-alkyl)(O⁻), —OP(O)(O-alkyl)(O-alkyl), —CO₂H, —C(O)O(alkyl), —CON(alkyl)(alkyl), —CONH(alkyl), —CONH₂, —C(O)(alkyl), —C(O)(phenyl), —C(O)(haloalkyl), —OC(O)(alkyl), —N(alkyl)(alkyl), —NH(alkyl), —N(alkyl)(alkylphenyl), —NH(alkylphenyl), —NHC(O)(alkyl), —NHC(O)(phenyl), —N(alkyl)C(O)(alkyl), and —N(alkyl)C(O)(phenyl).

As used in this specification and the appended claims, the articles “a,” “an,” and “the” include plural referents unless expressly and unequivocally limited to one referent.

The term “fatty acid” refers to any natural or synthetic carboxylic acid comprising an alkyl chain that may be saturated, monounsaturated, or polyunsaturated, and may have straight or branched chains. The fatty acid may also be substituted. “Fatty acid,” as used herein, includes short chain alkyl carboxylic acid including, for example, acetic acid, propionic acid, etc.

All numerical ranges herein include all numerical values and ranges of all numerical values within the recited range of numerical values.

The present disclosure relates to triester compounds, compositions, and methods of making the same. In certain embodiments, the present disclosure relates to biosynthetic triesters having one or more desirable physical properties, such as improved viscometrics, pour point, oxidative stability, hydrolytic stability, and/or viscosity index. In certain embodiments, the present disclosure relates to new methods of preparing triester compounds exhibiting such properties.

In certain embodiments, the compounds and compositions described herein comprise at least one compound selected from Formula I:

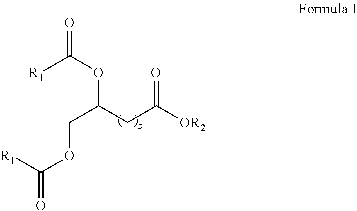

wherein

z is an integer selected from 0 to 15;

R₁, independently for each occurrence, is an optionally substituted alkyl that is saturated or unsaturated, and branched or unbranched; and

R₂ is selected from hydrogen and optionally substituted alkyl that is saturated or unsaturated, and branched or unbranched.

Also described herein are certain compounds which may be useful as lubricants, additives, or compound intermediates. In certain embodiments, such compounds are selected from compounds represented by Formula II:

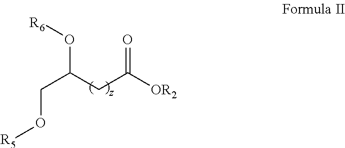

wherein

z is an integer selected from 0 to 15;

R₅ and R₆ are independently selected from hydrogen, —C(O)R₁, and an optionally substituted alkyl that is saturated or unsaturated, and branched or unbranched;

R₁ is, independently for each occurrence, an optionally substituted alkyl that is saturated or unsaturated, and branched or unbranched; and

R₂ is selected from hydrogen and optionally substituted alkyl that is saturated or unsaturated, and branched or unbranched.

In certain embodiments, the composition comprises at least one compound of Formula I or II, where R₁ is hydrogen.

The terms “chain” or “fatty acid chain” or “fatty acid chain residue,” as used with respect to the compounds of Formulas I-II, refer to one or more of the fatty acid residues incorporated in those compounds, e.g., R₁(O)O— and CH₂CH₂(CH₂)_{z}C(O)O— in Formulas I and II. CH₂CH₂(CH₂)_{z}C(O)O— in Formulas I and II may be referred to as the “base chain” or “base residue” or “fatty acid base chain.” Depending on the manner in which the compound is synthesized, the base organic acid or fatty acid residue may be the only residue that remains in its free-acid form after the initial synthesis. However, in certain embodiments, in an effort to alter or improve the properties of the compound, the free acid may be reacted with any number of substituents. For example, it may be desirable to react the free acid with alcohols, glycols, amines, or other suitable reactants to provide the corresponding ester, amide, or other reaction products. The base or base chain residue may also be referred to as tertiary or gamma (γ) chains.

The residues R₁C(O)O— in Formulas I and II may also be referred to as “caps” or “capping materials,” as it “caps” the base chain. In certain embodiments, the “caps” or “capping groups” are fatty acids. In certain embodiments, the capping group may be an organic acid residue. Similarly, the capping group may be an organic acid residue of general formula —OC(O)-alkyl, i.e., a carboxylic acid with an substituted or unsubstituted, saturated or unsaturated, and/or branched or unbranched alkyl as defined herein. In certain embodiments, the capping groups, regardless of size, are substituted or unsubstituted, saturated or unsaturated, and/or branched or unbranched. The caps or capping materials may also be referred to as the primary or alpha (α) chains.

Depending on the manner in which the triester is synthesized, the caps may be the only residues in the resulting triester that are unsaturated. In certain embodiments, it may be desirable to use saturated organic or fatty-acid caps to increase the overall saturation of the triester and/or to increase the resulting compound's stability. For example, in certain embodiments, it may be desirable to provide a saturated capped by epoxidizing, sulfurizing, and/or hydrogenating an unsaturated cap using any suitable methods available to those of ordinary skill in the art. Epoxidizing, sulfurizing, and/or hydrogenating may be used with various sources of the fatty-acid feedstock, which may include mono- and/or polyunsaturated fatty acids.

In certain embodiments, the triesters described herein can be prepared by epoxidizing one or more fatty acids or fatty acid esters having at least one terminal site of unsaturation. In certain embodiments, the epoxidizing may be accomplished using any of the methods generally known to those of ordinary skill in the art, such as using hydrogen peroxide and/or formic acid, or those methods involving one or more percarboxylic acids such as m-chloroperbenzoic acid, peracetic acid, or performic acid. Exemplary epoxidation methods also include those set forth in D. Swern, Organic Peroxides, Volume 2, 355-533, Interscience Publishers, 1971, which is incorporated by reference in its entirety for all purposes.

In certain embodiments, epoxidizing a fatty acid or fatty acid ester may provide for an intermediate compound, wherein the epoxide residue may be opened by reacting it with one or more compounds or compositions. For example, in certain embodiments, epoxidizing a terminally-unsaturated fatty acid or fatty acid ester (e.g., alkyl esters of 9-decenoic acid and 10-undecenoic acid) will provide a terminal epoxy group that may be opened to provide a mono-hydroxy compound or a vicinal dihydroxy compound. In certain embodiments, exposing a terminal epoxy fatty acid or fatty acid ester to aqueous acid conditions will provide a terminal vicinal dihydroxy compound. In certain embodiments, reacting an epoxy compound with an alcohol (e.g., fatty alcohol) under acidic conditions will provide a mono-hydroxy compound substituted with an alkoxy group. In certain embodiments, the epoxide residue may be opened by reacting the epoxy compound with a carboxylic acid (e.g., fatty acid) to provide the mono-hydroxy compound. In certain embodiments, compounds having free hydroxy groups may be acylated. In certain embodiments, fatty acid esters having terminal vicinal hydroxy groups may be acylated to provide the triester compounds described herein.

In certain embodiments, it may be desirable to provide a method of preparing a saturated capped triesters by hydrogenating one or more of the unsaturated caps using any suitable methods available to those of ordinary skill in the art. Hydrogenation may be used with various sources of the fatty-acid feedstock, which may include mono- and/or polyunsaturated fatty acids. Without being bound to any particular theory, in certain embodiments, hydrogenating the triester may help to improve the overall stability of the molecule. However, a fully-hydrogenated triester, such as triester with a larger fatty acid cap, may exhibit increased pour point temperatures. In certain embodiments, it may be desirable to offset any loss in desirable pour-point characteristics by using shorter, saturated capping materials, and/or branched capping materials.

As noted above, in certain embodiments, suitable terminally-unsaturated fatty acids, or esters thereof, for preparing the triesters described herein may include any mono- or polyunsaturated fatty acids, including natural or synthetic fatty acid sources. However, it may be desirable to source the fatty acids from a renewable biological feedstock. Suitable starting materials of biological origin may include plant fats, plant oils, plant waxes, animal fats, animal oils, animal waxes, fish fats, fish oils, fish waxes, algal oils and mixtures thereof. Other potential fatty acid sources may include waste and recycled food-grade fats and oils, fats, oils, and waxes obtained by genetic engineering, fossil fuel based materials and other sources of the materials desired.

In certain embodiments, the triester compounds described herein may be prepared from non-naturally occurring fatty acids derived from naturally occurring feedstocks. In certain embodiments, the compounds are prepared from synthetic fatty acid reactants derived from naturally occurring feedstocks such as vegetable oils. For example, the synthetic fatty acid reactants may be prepared by cleaving fragments from larger fatty acid residues occurring in natural oils such as triglycerides using, for example, a cross-metathesis catalyst and alpha-olefin(s). The resulting truncated fatty acid residue(s) may be liberated from the glycerine backbone using any suitable hydrolytic and/or transesterification processes known to those of skill in the art. An exemplary fatty acid reactant includes 9-decenoic acid, which may be prepared via the cross metathesis of an oleic acid residue with ethylene. In certain embodiments, the fatty acid reactant may comprise 10-undecenoic acid, which may be derived from the steam cracking (pyrolysis) of ricinoleic acid or an ester thereof, which may be sourced from castor oil.

In some embodiments, the compound comprises fatty-acid chains of varying lengths. In some embodiments, z is selected from 0 to 15, 0 to 12, 0 to 8, 0 to 6, 0 to 4, and 0 to 2. For example, in some embodiments, z is an integer selected from 0 to 15, 0 to 12, and 0 to 8. In some embodiments, z is an integer selected from 7 and 8. In some embodiments, z is selected from 1, 2, 3, 4, 5, 6, 7, 8, 9, 10, 11, 12, 13, 14, and 15.

In certain embodiments, R₅ and R₆, independently for each occurrence, are selected from hydrogen, —C(O)R₁, and an optionally substituted alkyl that is saturated or unsaturated, and branched or unbranched. In certain embodiments, R₅ and R₆ are hydrogen. In certain embodiments, R₅ and R₆ are independently selected from optionally substituted alkyl that is saturated or unsaturated, and branched or unbranched. In certain embodiments, R₅ and R₆ are independently selected from hydrogen and —C(O)R₁. In certain embodiments, R₅ and R₆ are independently selected from hydrogen and optionally substituted alkyl that is saturated or unsaturated, and branched or unbranched. In certain embodiments, R₅ and R₆ are independently selected from hydrogen and C₁-C₁₀ alkyl.

In some embodiments, R₁, independently for each occurrence, is an optionally substituted alkyl that is saturated or unsaturated, and branched or unbranched. In some embodiments, the alkyl group is a C₁ to C₄₀ alkyl, C₁ to C₂₂ alkyl, C₁ to C₁₅ alkyl, C₁ to C₁₇ alkyl, or C₉ to C₁₇ alkyl. In some embodiments, the alkyl group is a C₃ to Cᵢᵢ alkyl, C₅ to C₁₁ alkyl or C₉ to C₁₀ alkyl. In some embodiments, the alkyl group is selected from C₇ to C₁₇ alkyl, C₃ to C₁₃ alkyl, or C₅ to Cᵢᵢ alkyl. In some embodiments, each R₁ is independently selected from C₁ alkyl, C₂ alkyl, C₃ alkyl, C₄ alkyl, C₅ alkyl, C₆ alkyl, C₇ alkyl, C₈ alkyl, C₉ alkyl, C₁₀ alkyl, C₁₁ alkyl, C₁₂ alkyl, C₁₃ alkyl, C₁₄ alkyl, C₁₅ alkyl, C₁₆ alkyl, C₁₇ alkyl, C₁₈ alkyl, C₁₉ alkyl, C₂₀ alkyl, C₂₁ alkyl, C₂₂ alkyl, C₂₃ alkyl, and C₂₄ alkyl. In some embodiments, each R₁ is methyl. In some embodiments, R₁ is independently selected from C₁₃ to C₁₇ alkyl, such as from C₁₃ alkyl, C₁₅ alkyl, and C₁₇ alkyl.

It may be possible to manipulate one or more of the compounds' properties by altering the length of R₁ and/or its degree of saturation. However, the level of substitution on R₁ may also be altered to change or even improve the compounds' properties. Without being bound to any particular theory, it is believed that the presence of polar substituents on R₁, such as one or more hydroxy groups, may increase the viscosity of the compound, while adversely increasing pour point. Accordingly, in some embodiments, R₁ will be unsubstituted or optionally substituted with a group that is not hydroxyl.

In some embodiments, the compounds of Formulas I and II may be in their free-acid form, wherein R₂ is hydrogen. In some embodiments, R₂ is an optionally substituted alkyl that is saturated or unsaturated, and branched or unbranched. In some embodiments, the alkyl group is selected from C₁ to C₄₀, C₁ to C₂₂, C₃ to C₂₀, C₁ to C₁₈, or C₆ to C₁₂ alkyl. In some embodiments, R₂ is selected from C₃ alkyl, C₄ alkyl, C₈ alkyl, C₁₂ alkyl, C₁₆ alkyl, C₁₈ alkyl, and C₂₀ alkyl. For example, R₂ may be branched, such as isopropyl, isobutyl, or 2-ethylhexyl. In some embodiments, R₂ is a larger alkyl group, branched or unbranched, comprising C₁₂ alkyl, C₁₆ alkyl, C₁₈ alkyl, or C₂₀ alkyl. Such groups at the R₂ position may be derived from esterification of the free-acid compound using the Jarcol™ line of alcohols marketed by Jarchem Industries, Inc. of Newark, N.J., including Jarcol™ I-18CG, I-20, I-12, I-16, I-18T, and 85BJ. In some cases, R₂ may be sourced from certain alcohols to provide branched alkyls such as isostearyl and isopalmityl. It should be understood that such isopalmityl and isostearyl akyl groups may cover any branched variation of C₁₆ and C₁₈, respectively. For example, the compounds described herein may comprise highly-branched isopalmityl or isostearyl groups at the R₂ and R₃ positions, derived from the Fineoxocol® line of isopalmityl and isostearyl alcohols marketed by Nissan Chemical America Corporation of Houston, Tex., including Fineoxocol® 180, 180N, and 1600. Without being bound to any particular theory, in certain embodiments, it is believed that introducing large, highly-branched alkyl groups (e.g., isopalmityl and isostearyl) at the R₂ position of the compound may provide at least one way to increase the lubricant's viscosity, while substantially retaining or even reducing its pour point.

In certain embodiments, the fatty acid chains of the compounds described herein may be independently optionally substituted, wherein one or more hydrogens are removed and replaced with one or more of the substituents identified herein. Similarly, two or more of the hydrogen residues may be removed to provide one or more sites of unsaturation, such as a cis or trans double bond. In some embodiments, the chains may optionally comprise branched hydrocarbon residues.

In certain embodiments, the triester compounds herein may exhibit low temperature properties that make them attractive as lubricant base stocks or lubricant additives. In certain embodiments, the triesters may be combined with a base oil to provide a lubricant composition exhibiting excellent low temperature characteristics. In certain embodiments, the composition comprises a base oil and at least one triester compound. In certain embodiments, the composition further comprises at least one additive, such as those described herein. In certain embodiments, the triester comprises less than 20 wt. % of the composition, such as less than 15, 10, 8, or even 5 wt. % of the composition. In certain embodiments, the triester comprises about 0.01 to about 15 wt. % of the composition. In certain embodiments, the triester comprises about 0.1 to about 10 wt. % of the composition.

In certain embodiments, the composition may comprise an estolide base oil and at least one triester compound. In certain embodiments, the estolide base oil may comprise at least one compound of Formula III:

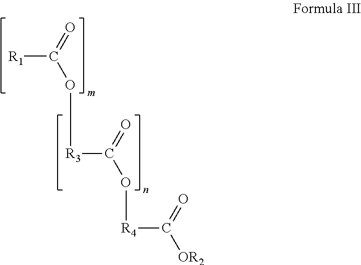

wherein
n is equal to or greater than 0;
m is equal to or greater than 1;
R₂ is selected from hydrogen and optionally substituted alkyl that is saturated or unsaturated, and branched or unbranched;
R₁ is selected from optionally substituted alkyl that is saturated or unsaturated, and branched or unbranched; and
R₃ and R₄, independently for each occurrence, are selected from optionally substituted alkylene that is saturated or unsaturated, and branched or unbranched.

In some embodiments, m is an integer selected from 1, 2, 3, 4, and 5. In some embodiments, m is 1. In some embodiments, n is an integer selected from 1, 2, 3, 4, 5, 6, 7, 8, 9, 10, 11, and 12. In some embodiments, R₁ comprises a group as previously defined herein. In certain embodiments, one or more R₃ differs from one or more other R₃ in a compound of Formula III. In some embodiments, one or more R₃ differs from R₄ in a compound of Formula III. In some embodiments, if the compounds of Formula III are prepared from one or more polyunsaturated fatty acids, it is possible that one or more of R₃ and R₄ will have one or more sites of unsaturation. In some embodiments, if the compounds of Formula III are prepared from one or more branched fatty acids, it is possible that one or more of R₃ and R₄ will be branched.

In certain embodiments, R₁ comprises C₁ to C₂₂ alkyl group that is branched or unbranched, and saturated or unsaturated. In certain embodiments, R₃ and R₄ are independently selected from a branched or unbranched C₁ to C₂₂ alkylene that is saturated or unsaturated. In certain embodiments, R₃ and R₄ are unbranched. In certain embodiments, R₃ and R₄ are saturated. In certain embodiments, R₁ comprises a C₉ to C₁₇ alkyl group. In certain embodiments, R₃ and R₄ are independently selected from C₉ to C₁₇ alkylene.

In certain embodiments, Applicant has discovered that triester compounds comprising terminal vicinal substituents exhibit surprising low temperature and viscometric properties. Without being bound to any particular theory, in certain embodiments it is believed that triesters comprising terminal vicinal substituents—and thus lacking a “hydrocarbon tail” on the base fatty acid residue—lower the crystallization temperature of the compound and, thus, the compound's pour point. It is also believed that providing branching of the acyl/alkoxy substituents (e.g., R₁, R₅ and/or R₆) and base ester residue (R₂) may further improve the cold temperature properties of the compound.

In some embodiments, the compounds and compositions described herein may exhibit viscosities less than about 55 cSt at 40° C. or less than about 45 cSt at 40° C., and/or less than about 12 cSt at 100° C. or less than about 10 cSt at 100° C. In some embodiments, compounds and compositions may exhibit viscosities less than about 40 cSt at 40° C. or less than about 30 cSt at 40° C., and/or less than about 8 cSt at 100° C. or less than about 6 cSt at 100° C. In some embodiments, the compounds and compositions may exhibit viscosities less than about 20 cSt at 40° C., and/or less than about 5 cSt at 100° C. In some embodiments, the compounds and compositions may exhibit viscosities within a range from about 15 cSt to about 25 cSt at 40° C., and/or about 3 cSt to about 6 cSt at 100° C. In some embodiments, the compounds and compositions may exhibit viscosities within a range from about 18 cSt to about 20 cSt at 40° C., and/or about 4 cSt to about 5 cSt at 100° C. In some embodiments, the compounds and compositions may exhibit viscosities of about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, or 55 cSt at 40° C. In some embodiments, the compounds and compositions may exhibit viscosities of about 1, 2, 3, 4, 5, 6, 7, 8, 9, 10, 11, 12, 13, 14, 15, 16, 17, 18, 19, 20, 25, or 30 cSt at 100° C.

In certain embodiments, the compounds may exhibit desirable low-temperature pour point properties. In some embodiments, compounds and compositions may exhibit a pour point lower than about −40° C., −50° C., −60° C., −70° C., or even −80° C. In some embodiments, the compound will have a pour point of about −40° C. to about −90° C., such as about −50° C. to about −60° C., −60° C. to about −70° C., or even −70° C. to about −80° C.

In addition, in certain embodiments, the compounds described herein may exhibit decreased Iodine Values (IV) when compared to compounds prepared by other methods. IV is a measure of the degree of total unsaturation of an oil, and is determined by measuring the amount of iodine per gram of compound (cg/g). In certain instances, oils having a higher degree of unsaturation may be more susceptible to creating corrosiveness and deposits, and may exhibit lower levels of oxidative stability. Compounds having a higher degree of unsaturation will have more points of unsaturation for iodine to react with, resulting in a higher IV. Thus, in certain embodiments, it may be desirable to reduce the IV of compounds in an effort to increase the oil's oxidative stability, while also decreasing harmful deposits and the corrosiveness of the oil.

In some embodiments, the compounds described have an IV of less than about 40 cg/g or less than about 35 cg/g. In some embodiments, the compounds will have an IV of less than about 30 cg/g, less than about 25 cg/g, less than about 20 cg/g, less than about 15 cg/g, less than about 10 cg/g, or less than about 5 cg/g. The IV of the compound may be reduced by decreasing the compound's degree of unsaturation. In certain embodiments, this may be accomplished by, for example, increasing the amount of saturated capping materials relative to unsaturated capping materials when synthesizing the compounds. Alternatively, in certain embodiments, IV may be reduced by hydrogenating compounds having unsaturated caps.

The present disclosure further relates to methods of making compounds according to Formulas I-II. By way of example, the reaction of an epoxy fatty ester with a fatty acid and/or aqueous acid may provide a mono- or di-hydroxy product that is useful as an intermediate to provide the ester products described herein.

As discussed in the schemes outlined further below, compound 102 represents a terminally-unsaturated fatty ester that may serve as the basis for preparing the compounds described herein.

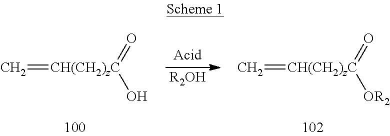

In Scheme 1, wherein z is an integer selected from 0 to 15, terminally-unsaturated fatty acid 100 may be esterified by any suitable procedure known to those of skilled in the art, such as acid-catalyzed reduction with alcohol R₂OH, to yield fatty ester 102. Other exemplary methods may include other types of Fischer esterification, such as those using Lewis acid catalysts such as BF₃.

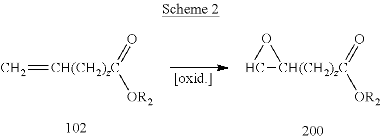

In Scheme 2, terminally-unsaturated fatty ester 102 may be contacted with an oxidant suitable for effecting epoxidation, such as hydrogen peroxide and formic acid, or a peracid such as mCPBA, to form epoxy ester 200.

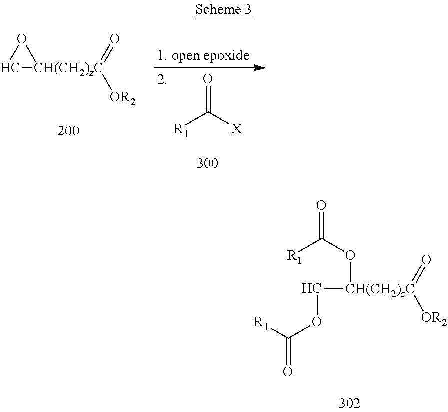

In Scheme 3, wherein z is an integer selected from 0 to 15, R₂ is an optionally-substituted alkyl that is saturated or unsaturated, and branched or unbranched, and R₁ is an optionally substituted alkyl group that is saturated or unsaturated, and branched or unbranched, epoxy ester 200 may be contacted with a compound or composition that will open the epoxide residue and provide the corresponding monohydroxy or dihydoxy variant, which may be isolated or generated in situ. For example, epoxy ester 200 may be contacted with an aqueous solution of acid, such as TfOH, to provide the dihydroxy fatty ester. Alternatively, epoxy ester 200 may be contacted with a fatty acid (such as octanoic acid) which will “cap” the compound by reacting with the epoxide residue to provide the monohydroxy variant. Subsequently, the monohydroxy or dihydroxy compound is contacted with electrophilic compound 300, where “x” is a leaving group (e.g., halide such as chlorine), to provide triester 302. In certain embodiments, electrophilic compound 300 is a fatty acid halide or fatty anhydride. Exemplary fatty acid halides include short-chain fatty acid chlorides such as hexanoyl and octanoyl chloride.

In certain embodiments, the compositions described herein may meet or exceed one or more of the specifications for certain end-use applications, without the need for conventional additives. For example, in certain instances, high-viscosity lubricants, such as those exhibiting a kinematic viscosity of greater than about 120 cSt at 40° C., or even greater than about 200 cSt at 40° C., may be desirable for particular applications such as gearbox or wind turbine lubricants. Prior-known lubricants with such properties typically also demonstrate an increase in pour point as viscosity increases, such that prior lubricants may not be suitable for such applications in colder environments. However, in certain embodiments, the counterintuitive properties of certain compositions described herein may make higher-viscosity compounds particularly suitable for such specialized applications.

Similarly, the use of prior-known lubricants in colder environments may generally result in an unwanted increase in a lubricant's viscosity. Thus, depending on the application, it may be desirable to use lower-viscosity oils at lower temperatures. In certain circumstances, low-viscosity oils may include those exhibiting a viscosity of lower than about 50 cSt at 40° C., or even about 40 cSt at 40° C. Accordingly, in certain embodiments, the low-viscosity compounds and compositions described herein may provide end users with a suitable alternative to high-viscosity lubricants for operation at lower temperatures.

In some embodiments, it may be desirable to prepare lubricant compositions comprising one or more triester compounds. For example, in certain embodiments, the compounds described herein may be blended with one or more additives selected from estolides, polyalphaolefins, synthetic esters, polyalkylene glycols, mineral oils (Groups I, II, and III), pour point depressants, viscosity modifiers, antioxidants, anti-corrosives, antiwear agents, detergents, dispersants, colorants, antifoaming agents, and demulsifiers. In addition, or in the alternative, in certain embodiments, the estolides described herein may be co-blended with one or more synthetic or petroleum-based oils to achieve the desired viscosity and/or pour point profiles. In certain embodiments, the compounds described herein also mix well with gasoline, so that they may be useful as fuel components or additives.

In all of the foregoing examples, the compounds described may be useful alone, as mixtures, or in combination with other compounds, compositions, and/or materials.

Methods for obtaining the novel compounds described herein will be apparent to those of ordinary skill in the art, suitable procedures being described, for example, in the examples below, and in the references cited herein.

### EXAMPLES

### Analytics

Nuclear Magnetic Resonance:

NMR spectra were collected using a Varian 300 spectrometer with an absolute frequency of 299.839 MHz at 297.1 K using CDCl₃ as the solvent. Chemical shifts were reported as parts per million from tetramethylsilane. The formation of a secondary ester link between fatty acids, as indicated by the presence of a vicinal methine proton, was verified with ¹H NMR by a multiplet peak between about 5.0 and 5.1 ppm.

Iodine Value (IV):

The iodine value is a measure of the degree of total unsaturation of an oil. IV is expressed in terms of centigrams of iodine absorbed per gram of oil sample. Therefore, the higher the iodine value of an oil the higher the level of unsaturation is of that oil. The IV may be measured and/or estimated by GC analysis. Where a composition includes unsaturated compounds other than compounds as set forth in Formula I-II, the compounds can be separated from other unsaturated compounds present in the composition prior to measuring the iodine value of the constituent estolides. For example, if a composition includes unsaturated fatty acids or triglycerides comprising unsaturated fatty acids, these can be separated from the compounds present in the composition prior to measuring the iodine value for the one or more compounds.

IV Calculation:

The iodine value is estimated by the following equation based on ASTM Method D97 (ASTM International, Conshohocken, Pa.):

$$IV = \sum 100 \times \frac{{A}_{f} \times M {W}_{I} \times db}{M {W}_{f}}$$

A_{f}=fraction of fatty compound in the sample
MW_{I}=253.81, atomic weight of two iodine atoms added to a double bond
db=number of double bonds on the fatty compound

MW_{f}=molecular weight of the fatty compound

Acid Value:

The acid value is a measure of the total acid present in an oil. Acid value may be determined by any suitable titration method known to those of ordinary skill in the art. For example, acid values may be determined by the amount of KOH that is required to neutralize a given sample of oil, and thus may be expressed in terms of mg KOH/g of oil.

The properties of exemplary compounds and compositions described herein are identified in the following examples and tables.

Other Measurements:

Except as otherwise described, pour point is measured by ASTM Method D97-96a, cloud point is measured by ASTM Method D2500, viscosity/kinematic viscosity is measured by ASTM Method D445-97, viscosity index is measured by ASTM Method D2270-93 (Reapproved 1998), specific gravity is measured by ASTM Method D4052, flash point is measured by ASTM Method D92, evaporative loss is measured by ASTM Method D5800, vapor pressure is measured by ASTM Method D5191, and acute aqueous toxicity is measured by Organization of Economic Cooperation and Development (OECD) 203.

### Example 1

Under argon in a three-neck 2 L roundbottom flask equipped with a condenser and mechanical stirrer and placed in a sand bath was added 2-ethylhexanol (10 eq, 0.33 mol, 43 g, 51.6 mL) and 10-undecenoic acid (1.00 eq, 6 g, 0.033 mol). The reaction mixture was stirred at room temperature for 5 minutes to achieve complete dissolution. Methanesulfonic acid (0.1 eq, 0.32 g, 0.21 mL, 0.0033 mol) was then added, and the mixture was stirred at 85° C. and monitored by TLC until completion (approx. 1.5 hrs). The reaction mixture was then cooled to ambient temperature, and under stirring was added 50% aqueous sodium bicarbonate (20 mL). The organic layer was extracted with EtOAc (3×) and concentrated by rotary evaporation. The resulting solution was distilled at 170-200° C. under house vacuum to remove excess 2-ethylhexanol, yielding the desired 10-undecenoic acid 2-ethylhexyl ester in quantitative yield.

### Example 2

Under argon in a three-neck 2 L roundbottom flask equipped with a magnetic stir bar was added 10-undecenoic acid 2-ethylhexyl ester (1.00 eq, 3 g, 0.010 mol) prepared according to the method set forth in Example 1, and 25 mL of dichloromethane. Under stirring at 45° C., 75% mCPBA (2.2 eq, 5 g, 0.022 mol) was slowly added over 30 minutes. Stirring of the reaction at 45° C. was continued for 1.5-2 hrs until the reaction was completed as confirmed by TLC. The reaction mixture was filtered over filter paper, and the filtrate was carefully washed with 10% aqueous sodium bicarbonate. The organic layer was washed with water (2×), dried over MgSO₄, and concentrated under rotary evaporation to provide the crude epoxy ester product in quantitative yield.

### Example 3

Under argon in a three-neck 2 L roundbottom flask equipped with a magnetic stir bar was added epoxy undecanoic acid 2-ethylhexyl ester (1.00 eq, 3.12 g, 0.010 mol) prepared according to the method set forth in Example 2, and 25 mL of THF in 25 mL of water. Under stirring, 1 mL of TfOH was slowly added to the reaction mixture at rt. Stirring was continued and the reaction was monitored by TLC until completion (apprx. 5 hrs). The reaction mixture was quenched with 50% aqueous sodium bicarbonate, and stirring was continued for an additional 15 mins. The organic layer was then separated, and additional washes of the organic layer with 50% aqueous sodium bicarbonate were continued until the organic layer exhibited a pH of 7 to 8. The organic layer was then dried over MgSO₄, and concentrated under rotary evaporation to provide the crude dihydroxy fatty ester product (oily white solid).

### Example 4

Crude dihydroxy fatty ester (1.00 eq, 850 mg, 2.57 mmol) prepared according to the method set forth in Example 3, and pyridine (8 mL) were added to a 2-neck roundbottom flask affixed with a condenser. Acetic anhydride (3 eq, 787 mg, 0.73 mL, 7.71 mmol) was added via syringe, and the reaction was refluxed under stirring for 1.5 hrs. The reaction mixture was allowed to cool to ambient temperature, and then a cold 10% aqueous sodium bicarbonate solution (15 mL) was added and the mixture was allowed to stir for 10-15 minutes. Aliquots of 50% aqueous sodium bicarbonate solution were added to the stirred organic layer until the aqueous layer tested as basic using litmus paper. The organic layer was diluted with 20 mL of EtOAC and was then washed with aliquots of 10% aqueous copper sulfate until the color of the aqueous layer indicated the absence of pyridine (change from purple to blue). The organic layer was dried over MgSO₄, and concentrated via rotary evaporation to obtain the crude triester. The crude product was purified by affinity chromatography (SiO₂ and 10% EtOAc/hexanes) to provide the pure triester product, as confirmed by ¹H NMR. The triester exhibited a freezing point of about −56° C. to about −60° C., and a kinematic viscosity of less than 10 cSt when measured at 100° C.

### Example 5

Crude dihydroxy ester (1.00 eq, 33 g, 0.1 mol) prepared according to the method set forth in Example 3, and pyridine (250 mL) were added to a 2-neck roundbottom flask affixed with a condenser. Isobutyric anhydride (3 eq, 47.46 g, 50 mL, 0.3 mol) was added via syringe, and the reaction was refluxed under stirring for 1.5 hrs. The reaction mixture was allowed to cool to ambient temperature, and then a cold 10% aqueous sodium bicarbonate solution (15 mL) was added and the mixture was allowed to stir for 10-15 minutes. Aliquots of 50% aqueous sodium bicarbonate solution were added to the stirred organic layer until the aqueous layer tested as basic using litmus paper. The organic layer was diluted with 100 mL of EtOAc and was then washed with aliquots of 10% aqueous copper sulfate until the color of the aqueous layer indicated the absence of pyridine (change from purple to blue). The organic layer was dried over MgSO₄, and concentrated via rotary evaporation to obtain the crude triester. The crude product was purified by affinity chromatography (SiO₂ and 10% EtOAc/Hexanes) to provide the pure triester product, as confirmed by ¹H NMR. The triester exhibited a freezing point of about −70° C. to about −78° C., and a kinematic viscosity of less than 10 cSt when measured at 100° C.

### Example 6

Compounds are prepared according to the method set forth in Example 5, except isobutyric anhydride is replaced with an equal molar amount of hexanoic anhydride to provide the desired triester product.

### Example 7

Compounds are prepared according to the method set forth in example 5, except decenoyl chloride is replaced with an equal molar amount of hexanoic anhydride to provide the desired triester product.

### Example 8

Triesters are prepared according to the methods set forth in Examples 1-7, except the 2-ethylhexanol esterifying alcohol is replaced with various alcohols including those set forth below, which may be saturated or unsaturated and unbranched or substituted with one or more alkyl groups selected from methyl, ethyl, propyl, isopropyl, butyl, isobutyl, sec-butyl, tert-butyl, pentyl, isopentyl, neopentyl, hexyl, isohexyl, and the like, to form a branched residue at the R₂ position:

TABLE 1

| Alcohol | R₂ Substituents |
|---|---|
| C₁ alkanol | methyl |
| C₂ alkanol | ethyl |
| C₃ alkanol | n-propyl, isopropyl |
| C₄ alkanol | n-butyl, isobutyl, sec-butyl |
| C₅ alkanol | n-pentyl, isopentyl neopentyl |
| C₆ alkanol | n-hexyl, 2-methyl pentyl, 3- |
| | methyl pentyl, 2,2-dimethyl |
| | butyl, 2,3-dimethyl butyl |
| C₇ alkanol | n-heptyl and other structural |
| | isomers |
| C₈ alkanol | n-octyl and other structural |
| | isomers |
| C₉ alkanol | n-nonyl and other structural |
| | isomers |
| C₁₀ alkanol | n-decanyl and other structural |
| | isomers |
| C₁₁ alkanol | n-undecanyl and other structural |
| | isomers |
| C₁₂ alkanol | n-dodecanyl and other structural |
| | isomers |
| C₁₃ alkanol | n-tridecanyl and other structural |
| | isomers |
| C₁₄ alkanol | n-tetradecanyl and other |
| | structural isomers |
| C₁₅ alkanol | n-pentadecanyl and other |
| | structural isomers |
| C₁₆ alkanol | n-hexadecanyl and other |
| | structural isomers |
| C₁₇ alkanol | n-heptadecanyl and other |
| | structural isomers |
| C₁₈ alkanol | n-octadecanyl and other |
| | structural isomers |
| C₁₉ alkanol | n-nonadecanyl and other |
| | structural isomers |
| C₂₀ alkanol | n-icosanyl and other structural |
| | isomers |
| C₂₁ alkanol | n-heneicosanyl and other |
| | structural isomers |
| C₂₂ alkanol | n-docosanyl and other structural |
| | isomers |

1-27. (canceled)

28. At least one compound of Formula II: 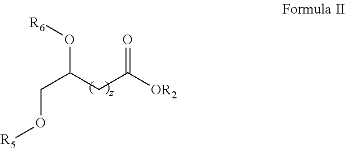
wherein
z is an integer selected from 0 to 15;
R₅ and R₆ are independently selected from hydrogen, —C(O)R₁, and an optionally substituted alkyl that is saturated or unsaturated, and branched or unbranched;
R₁ is, independently for each occurrence, an optionally substituted alkyl that is saturated or unsaturated, and branched or unbranched; and
R₂ is selected from hydrogen and optionally substituted alkyl that is saturated or unsaturated, and branched or unbranched.

29. (canceled)

30. The at least one compound according to claim 28, wherein z is an integer selected from 0 to 8.

31. The at least one compound according to claim 30, wherein z is an integer selected from 7 and 8.

32. The at least one compound according to claim 28, wherein R₅ and R₆ are independently selected from hydrogen and —C(O)R₁.

33. The at least one compound according to claim 32, wherein R₅ and R₆ are —C(O)R₁.

34. The at least one compound according to claim 33, wherein R₁, independently for each occurrence, is selected from an optionally substituted C₁ to C₁₈ alkyl that is saturated or unsaturated, and branched or unbranched.

35. (canceled)

36. (canceled)

37. The at least one compound according to claim 34, wherein R₁, independently for each occurrence, is selected from C₉ and C₁₀ alkyl.

38. The at least one compound according to claim 34, wherein R₁ is saturated for each occurrence.

39. (canceled)

40. The at least one compound according to claim 37, wherein R₁ is terminally unsaturated for each occurrence.

41. (canceled)

42. The at least one compound according to claim 34, wherein R₁ is branched for each occurrence.

43. The at least one compound according to claim 34, wherein R₁ is isopropyl.

44. (canceled)

45. The at least one compound according to claim 34, wherein R₁ is n-nonyl

46. The at least one compound according to claim 34, wherein R₁ is n-decanyl

47. The at least one compound according to claim 34, wherein R₁ is unsubstituted for each occurrence.

48. The at least one compound according to claim 28, wherein R₅ and R₆ are hydrogen.

49. The at least one compound according to claim 28, wherein R₅ and R₆ are independently selected from hydrogen and optionally substituted alkyl that is saturated or unsaturated, and branched or unbranched.

50. The at least one compound according to claim 49, wherein R₅ and R₆ are independently selected from hydrogen and C₁-C₁₀ alkyl.

51. (canceled)

52. (canceled)

53. The at least one compound according to claim 28, wherein R₂ is selected from optionally substituted C₁ to C₁₈ alkyl that is saturated or unsaturated, and branched or unbranched.

54. (canceled)

55. (canceled)

56. The at least one compound according to claim 28, wherein R₂ is selected from optionally substituted C₆ to C₁₂ alkyl that is saturated or unsaturated and branched or unbranched.

57. The at least one compound according to claim 56, wherein R₂ is 2-ethylhexyl.

58. A method comprising:
selecting a first composition, wherein said first composition exhibits an initial pour point; and
contacting the first composition with at least one triester having terminal vicinal acyl groups to provide a second composition,
wherein the second composition exhibits a resulting pour point that is lower than the initial pour point.

59-76. (canceled)
